(19) United States
(12) Patent Application Publication
(10) Pub. No.: US 2026/0315224 A1
(43) Pub. Date: Oct. 15, 2026

(54) Catheter Anchoring to Control Inflow Placement of Balloon Expandable Prosthetic Heart Valve

(71) Applicant: St. Jude Medical, Cardiology Division, Inc., St. Paul, MN (US)

(72) Inventor: William H. Peckels, Houlton, WI (US)

(73) Assignee: St. Jude Medical, Cardiology Division, Inc., St. Paul, MN (US)

(21) Appl. No.: 19/643,264

(22) Filed: Apr. 9, 2026

Related U.S. Application Data
(60) Provisional application No. 63/786,658, filed on Apr. 10, 2025.

(51) Int. Cl. A61F 2/24

(52) U.S. Cl. CPC A61F 2/2433

(57) ABSTRACT

A prosthetic heart valve implantation system, including a collapsible and expandable prosthetic heart valve, a delivery system for delivering and deploying the collapsible and expandable prosthetic heart valve, and an anchoring system operably coupled to the delivery system. The delivery system includes a delivery catheter and an inflatable balloon at a distal end portion of the delivery catheter. The anchoring system includes an anchor having a compressed or stowed condition, and an expanded or deployed condition, the anchor being configured engage with patient anatomy in the expanded or deployed configuration of the anchor to anchor the delivery system relative to patient anatomy to restrict axial movement of the delivery catheter during inflation of the inflatable balloon.

### CROSS-REFERENCE TO RELATED APPLICATIONS

This application claims the benefit of the filing date of U.S. Provisional Patent Application No. 63/786,658, filed Apr. 10, 2025, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

### SUMMARY OF THE DISCLOSURE

According to an aspect of the disclosure, a prosthetic heart valve implantation system includes: a collapsible and expandable prosthetic heart valve; a delivery system for delivering and deploying the collapsible and expandable prosthetic heart valve, the delivery system including a delivery catheter and an inflatable balloon at a distal end portion of the delivery catheter; and an anchoring system operably coupled to the delivery system, the anchoring system including an anchor having a compressed or stowed condition, and an expanded or deployed condition, the anchor being configured engage with patient anatomy in the expanded or deployed configuration of the anchor to anchor the delivery system relative to patient anatomy to restrict axial movement of the delivery catheter during inflation of the inflatable balloon.

In one example, the anchoring system includes: at least one control wire coupled to the anchor, wherein applying tension to the control wire causes the anchor to either (i) move from the compressed or stowed configuration to the expanded or deployed configuration, or (ii) move from the expanded or deployed configuration to the compressed or stowed configuration.

In one example, the anchoring system includes: a tensioning mechanism disposed in a handle of the delivery system, the tensioning mechanism includes a plurality of springs coupled to the at least one control wire so as to apply a uniform tension to the at least one control wire.

In one example, the anchor has a frustoconical or curved frustoconical shape defining a first end and a second end, the anchor being shape-set to the expanded or deployed configuration such that the anchor has the frustoconical or curved frustoconical shape in the absence of applied forces and, wherein upon application of tension from the at least one control wire, the anchor transitions from the expanded or deployed condition to the compressed or stowed configuration.

In one example, the first end of the anchor, in the expanded or deployed configuration, is sized and shaped to contact and exert a force on the patient anatomy to anchor the delivery system relative to the patient anatomy.

In one example, the anchor is formed of a shape memory material, the shape memory material being a nickel-titanium alloy.

In one example, the anchor includes a base section and a gripping section, the gripping section including a plurality of frictional elements, wherein the anchor is shape-set to the expanded or deployed configuration and, upon application of tension from the at least one control wire, the anchor compresses from the expanded or deployed configuration to the compressed or stowed configuration.

In one example, the frictional elements, in the expanded or deployed configuration, are sized and shaped to frictionally engage with the patient anatomy to anchor the delivery system relative to the patient anatomy.

In one example, the frictional elements are angled in an antegrade direction relative to the gripping section so as to prevent the frictional elements from inadvertently snaring tissue during delivery.

In one example, the anchor comprises a textured portion of the delivery catheter and the anchoring system further comprises a retractable sheath configured to be translated relative to the delivery catheter to either (i) uncover the anchor for frictional engagement with the patient anatomy or (ii) cover the anchor to prevent frictional engagement with the patient anatomy.

In one example, the anchoring system includes a plurality of anchors, each of the plurality of anchors including a base section and a gripping section, the gripping section including a plurality of frictional elements, wherein the plurality of anchors are shape-set to the compressed or stowed configuration such that, upon application of tension from the at least one control wire, the anchor is expanded from the compressed or stowed condition to the expanded or deployed configuration.

In one example, the frictional elements, in the expanded or deployed configuration, are sized and shaped to frictionally engage with the patient anatomy to anchor the delivery system relative to the patient anatomy.

In one example, the frictional elements are angled in an antegrade direction relative to the gripping section so as to prevent the frictional elements from inadvertently snaring tissue during delivery.

In one example, the anchor comprises a single helix formed of a single wire or a multi-filament wire.

In one example, the anchor comprising the single helix formed of the single wire or the multi-filament wire defines a first end, a second end, and a coiled section between the first end and the second end, wherein the anchor is shape-set to the expanded or deployed configuration such that, upon application of tension from the at least one control wire, the anchor transitions from the expanded or deployed condition to the compressed or stowed configuration.

Another aspect of the disclosure provides a method of implanting a collapsible and expandable prosthetic heart valve into a patient, the method including: advancing a delivery catheter, while the prosthetic heart valve is in a collapsed condition and mounted on a balloon of the delivery catheter, through vasculature of the patient until the collapsed prosthetic heart valve is positioned adjacent to or within a native valve annulus of the patient; while the collapsed prosthetic heart valve is positioned adjacent to or within the native valve annulus, anchoring the delivery catheter to the vasculature to restrict axial movement of the delivery catheter relative to patient anatomy; and inflating the balloon, while the delivery catheter is anchored to the vasculature, to expand the collapsible and expandable prosthetic heart valve for deployment into the native valve annulus.

In one example, anchoring the delivery catheter includes expanding or deploying an anchoring system to engage with the vasculature.

In one example, the method further includes applying or releasing tension on or more control wires that are coupled to an anchor of the anchoring system to expand or deploy the anchor of the anchoring system.

In one example, the anchoring system is in a compressed or stowed configuration during advancing of the delivery catheter.

In one example, the method further includes releasing the anchoring system from the patient anatomy after the prosthetic heart valve is deployed in the native valve annulus.

In one example, the method further includes deflating the balloon; and retracting the delivery catheter from the patient.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the handle of the delivery system shown in FIG. 4.

FIG. 6 is an enlarged view of a distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the inflation system of FIG. 8.

FIG. 10 is a perspective view of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12 is a highly schematic illustration portions of a delivery system in the aortic arch during a valve replacement procedure.

FIG. 13 illustrates a delivery system incorporating an anchoring system according to one or more aspects of the disclosure.

FIGS. 13A-C depict a tensioning mechanism of an anchoring system according to one or more aspects of the disclosure.

FIG. 14 illustrates a delivery system incorporating an anchoring system according to one or more aspects of the disclosure.

FIG. 14A is an anchor of the anchoring system of FIG. 14.

FIG. 15 illustrates a delivery system incorporating an anchoring system according to one or more aspects of the disclosure.

FIG. 16 illustrates a delivery system incorporating an anchoring system according to one or more aspects of the disclosure.

FIG. 17 illustrates a delivery system incorporating an anchoring system according to one or more aspects of the disclosure.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

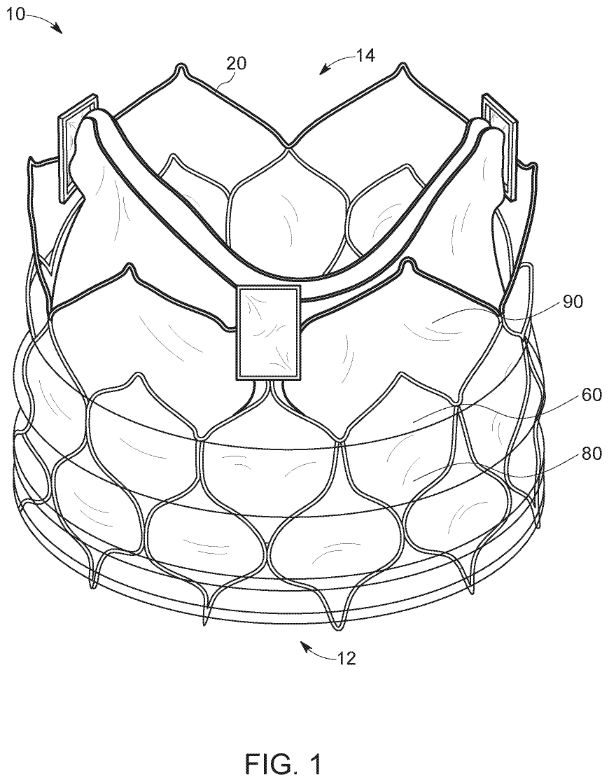

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

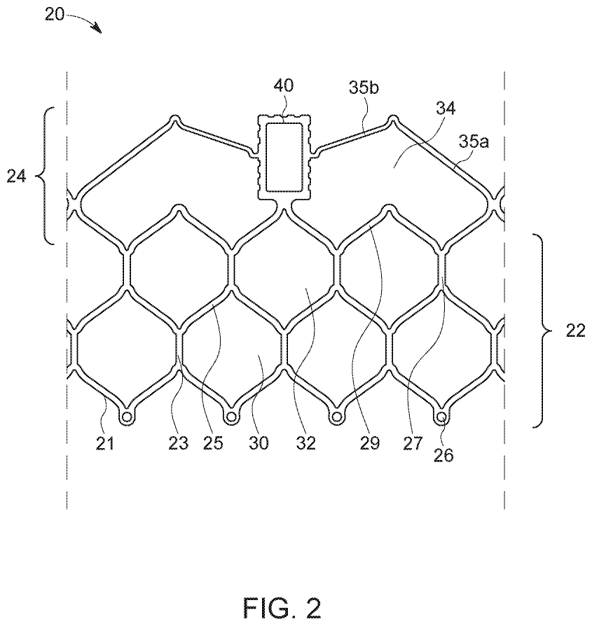

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g. fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Provisional Patent Application No. 63/579,378, filed Aug. 29, 2023 and titled “TAVI Deployment Accuracy-Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g. struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

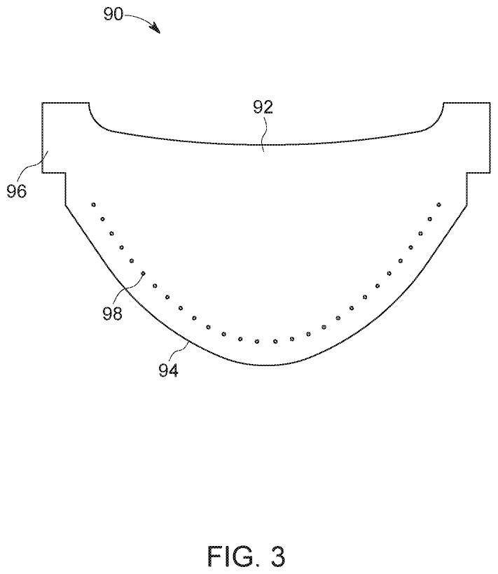

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g. via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Leaflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

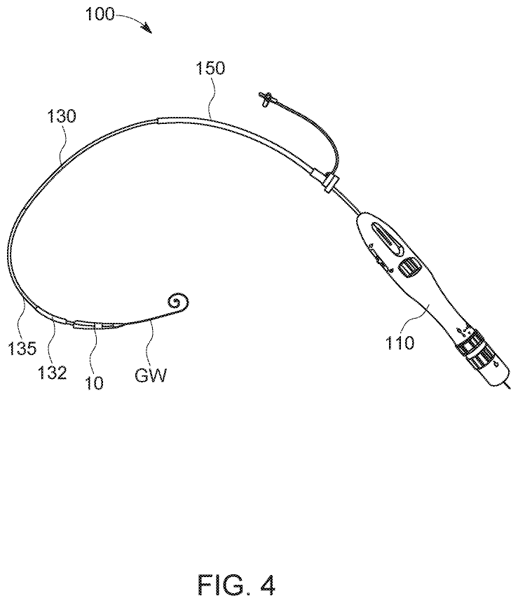

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

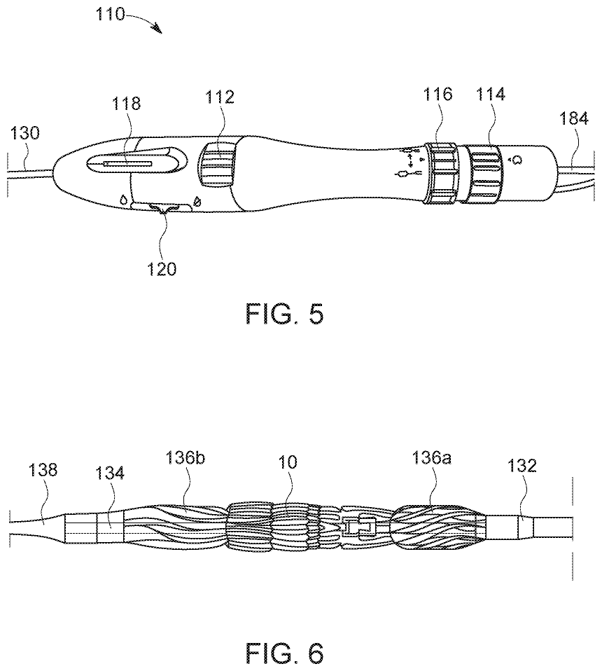

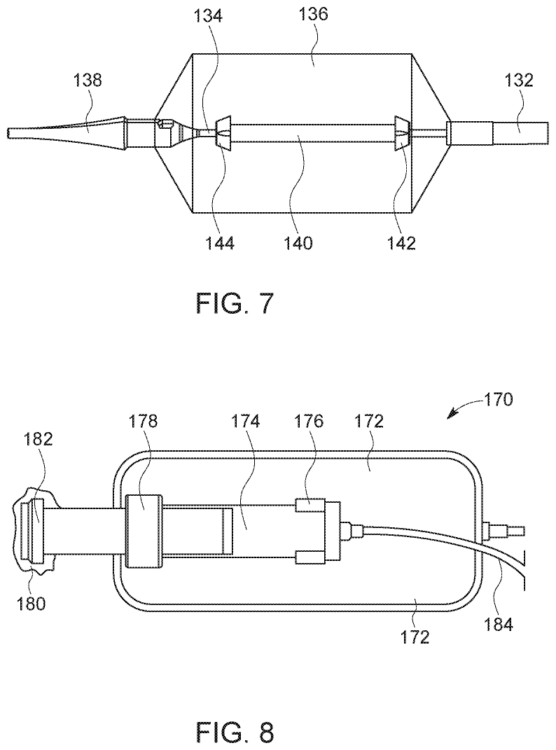

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135. In some examples, the handle may include a window 118 that allows viewing of an indicator that corresponds to the amount of catheter deflection. For example, a carrier to which the indicator is attached may be attached to the steering wire. In some examples, when there is minimum (or zero) tension on the steering wire, the indicator is at the far distal position within window 118, but as deflection is actuated, for example by drawing a carrier proximally (and tensioning the steering wire as the carrier draws proximally), the indicator will move proximally along window 118, giving the user a readily-apparent indication of the amount of deflection applied to the catheter at any given moment.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads (or another component, such as individual extensions, which may be cylindrical extensions that fit between internal threads of the actuator) of a carriage that is coupled to an inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it may have a small total range of motion, including for example between about 2 mm and about 15 mm of range of motion, including about 7.5 mm range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Provisional Patent Application No. 63/382,812, filed Nov. 8, 2022 and titled “Prosthetic Heart Valve Delivery and Trackability,” the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. In some embodiments, the balloon actuator 120 may take the form of a “momentary switch” in which pushing the balloon actuator 120 forward engages inflation, pulling the balloon actuator 120 proximally engages deflation, and releasing the balloon actuator 120 pauses inflation. This particular example of functionality may allow the physician to precisely control the amount of fluid dispensed while reducing the occurrence of over-or under-inflation, for example because the system automatically pauses inflation when the switch is released. The physical form factor of the balloon actuator 120 may be any suitable desired form factor, including for example a rocker switch, a push button, etc. In some embodiments a second balloon actuator or button may be provided, either on the balloon actuator 120 or elsewhere on the handle 110, with the second balloon actuator allowing for a change (e.g. increase or decrease) in the rate of inflation, for example to a pre-programmed faster or slower rate of inflation. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

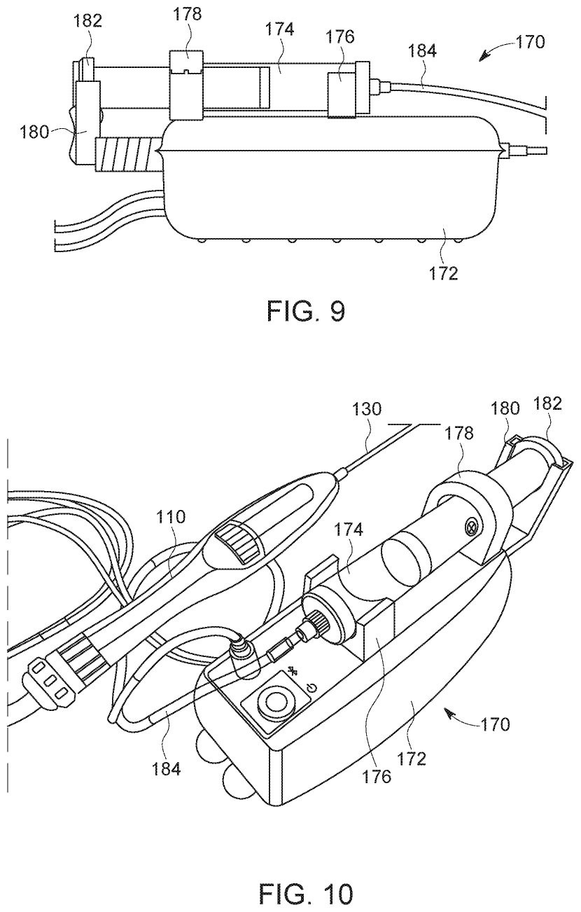

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g. from AC mains or another component with which the cable is coupled) and/or transmission of data, information, control commands, etc. For example, one cable may couple the housing 172 to handle 110 so that controls on the handle 110 (e.g. balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. patent application Ser. No. 18/311,458, the disclosure of which is hereby incorporated by reference herein.

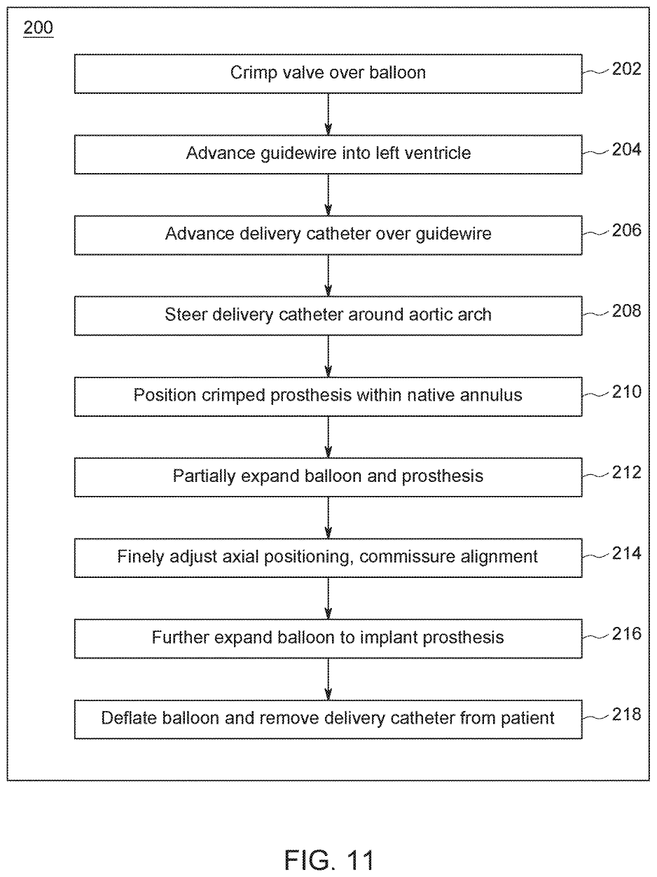

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g. by actuating axial alignment actuator 116) and/or the rotational orientation of the prosthetic heart valve 10 relative to the native aortic valve may be finely adjust (e.g. by actuating commissure alignment actuator 114). When the desired axial alignment is achieved and the desired rotational alignment (e.g. rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

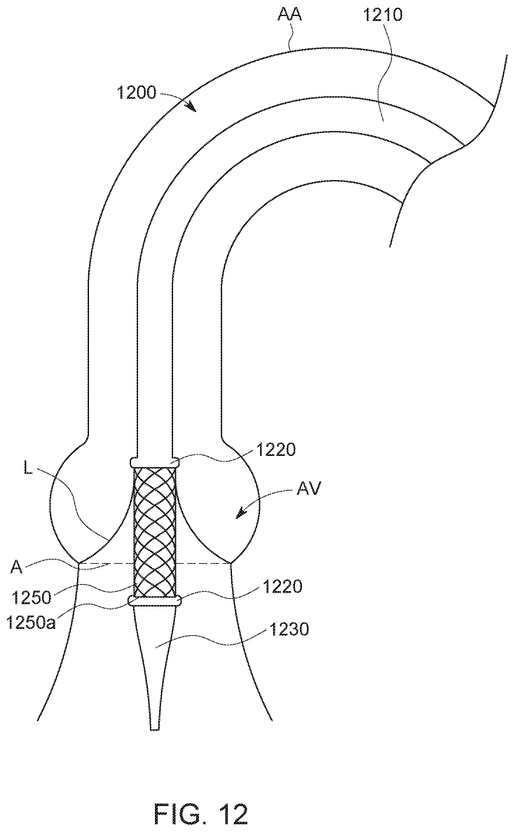

FIG. 12 is a highly schematic illustration of portions of a delivery system 1200 in the aortic arch AA during a valve replacement procedure. As shown, the delivery system 1200 can include a delivery catheter 1210 (e.g., delivery catheter 130 described above, including one or more of outer catheter 132, inner catheter 134, and/or steering catheter 135), a balloon 1220 (e.g., balloon 136 described above), and an atraumatic distal tip 1230 (e.g., atraumatic distal tip 138 described above).

In FIG. 12, the delivery catheter 1210 is illustrated passing through the aortic arch AA as part of a retrograde transfemoral transcatheter aortic valve replacement procedure. In the view of FIG. 12, a collapsed prosthetic heart valve 1250 is being advanced into a space between the native aortic valve leaflets L and within a native aortic valve annulus A of the native aortic valve AV. The dashed line in FIG. 12 represents the basal plane of the native aortic valve annulus A, which may represent a virtual plane passing through the points at which the bases of the aortic valve leaflets L attach to the aortic valve annulus A. Prosthetic heart valve 1250 may be any suitable collapsible and expandable prosthetic heart valve, such as prosthetic heart valve 10. However, the disclosure provided herein may be particularly relevant to balloon-expandable prosthetic heart valves.

As described above in FIG. 11 and with respect to step 210, the delivery catheter 1210 is advanced until the prosthetic heart valve 1250, while still crimped or collapsed over balloon 1220, is positioned within the native aortic valve annulus A. Proper positioning of the prosthetic heart valve 1250 within the native aortic valve annulus A can reduce risk for a post operative pacemaker implantation, paravalvular leak (PVL), and interaction with the anterior mitral valve leaflet. In some procedures, it may be desirable to implant the prosthetic heart valve 1250 such that the inflow edge 1250a aspect of the prosthetic heart valve 1250 is positioned generally between about 0 mm and about 8 mm subannularly with respect to the basal plane of the native aortic valve annulus A. See, e.g., Kim, WK., Renker, M., Doerr, O. et al. Impact of implantation depth on outcomes of new-generation balloon-expandable transcatheter heart valves. Clin Res Cardiol 110, 1983-1992 (2021), the disclosure of which is hereby incorporated by reference herein.

With reference again to the flowchart of FIG. 11, once the prosthetic heart valve 1250 is positioned within the native aortic valve annulus A, the balloon 1220 is expanded to implant the prosthetic heart valve 1250 in the native aortic valve AV. During inflation of the balloon 1220, however, the axial position of the prosthetic heart valve 1250, and particularly the inflow edge thereof, may change relative to the basal plane for at least three reasons. A first reason for such movement is that the frame (e.g., frame 20 described above) of the prosthetic heart valve 1250 may axially foreshorten as the prosthetic heart valve 1250 radially expands. However, the design of frame may largely limit the axial foreshortening to the outflow edge of the prosthetic heart valve 1250, with the inflow edge undergoing only a small amount of axial foreshortening during radial expansion. A second reason for such movement is that the delivery system 1200, including the delivery catheter 1210 itself, may tend to move as the balloon 1220 inflates. For example, inflation media flowing rapidly through the delivery catheter 1210 toward the balloon 1220 may tend to push the distal end of the delivery catheter 1210, including the ballon 1220, in the direction opposite to flow of the inflation media. In another example, positioning of the delivery catheter 1210 in the aortic arch prior to inflation can result in axial movement. In this regard, conformity of the delivery catheter 1210 and high calcification of the native annulus can result in the delivery catheter 1210 being positioned axially centrally relative to the valve, resulting in axial instability during inflation. A third reason for such movement is that inadequate or loss of rapid cardiac pacing may destabilize deployment during inflation, with the inadequate or loss of rapid cardiac pacing resulting in an increase in pulse pressure delta (a difference between systolic and diastolic blood pressure). Generally, where the pulse pressure delta is below 15 mmhg, deployment during inflation remains stable, while a pulse pressure delta of 15 mmhg or greater can result in the destabilization mentioned above. In the illustrated examples, this may result in antegrade movement of the balloon 1220 during inflation, causing the entire prosthetic heart valve 1250 mounted on the balloon 1220 to move in the antegrade direction. Thus, while the prosthetic heart valve 1250 may be optimally positioned (e.g., between 0 and 8 mm subannularly with respect to the basal plane) prior to inflation of the balloon 1220, the antegrade movement of the balloon 1220 mentioned above may disrupt that positioning, resulting in implantation in a suboptimal position.

One aspect of the present disclosure provides an anchoring device that allows the delivery catheter to resist the antegrade motion during the inflation and will simplify and standardize the implantation within the optimal zone relative to the native aortic valve annulus A.

Advantageously, implementation of the anchoring device stabilizes the delivery catheter 1210 so that the balloon 1220 does not move axially during inflation (e.g. antegrade along the ascending aortic arch AA and native aortic valve annulus A) to provide a stable position for the expanding prosthetic heart valve 1250 during valve deployment. Minimizing or eliminating antegrade movement of the delivery system catheter 1210 can thus reduce overall movement of the prosthetic heart valve 1250 during inflation, resulting in more predictable and more precise placement of the prosthetic heart valve 1250, and particularly of the inflow edge of the prosthetic heart valve 1250 relative to the basal plane of the native aortic valve annulus A.

Further advantageously, implementation of the anchoring device can prevent destabilization that may occur due to inadequate or loss of rapid cardiac pacing. Stabilizing the delivery catheter during delivery and deployment may reduce the pacing burden that is typically required to keep pulse pressure delta below 15 mmhg. In this regard, stable delivery and deployment can be achieved at a pulse pressure delta greater than 15 mmhg, such as 20 or 25 mmhg.

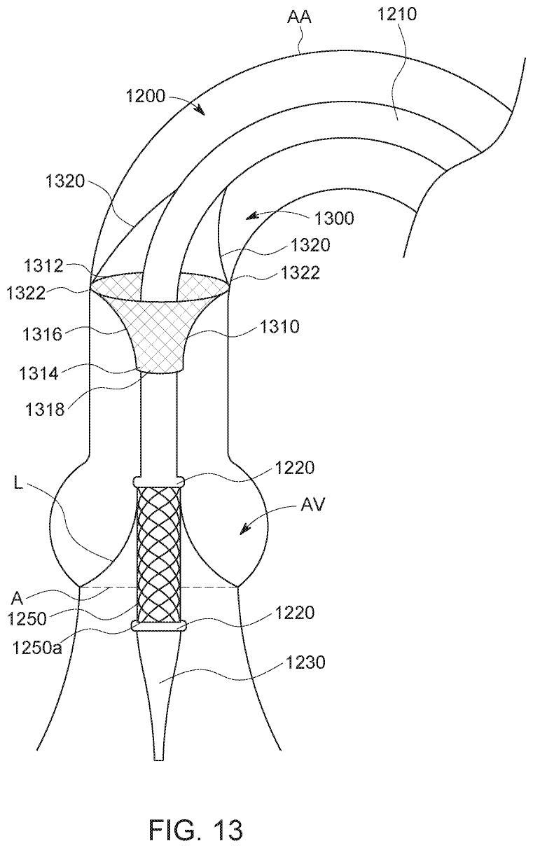

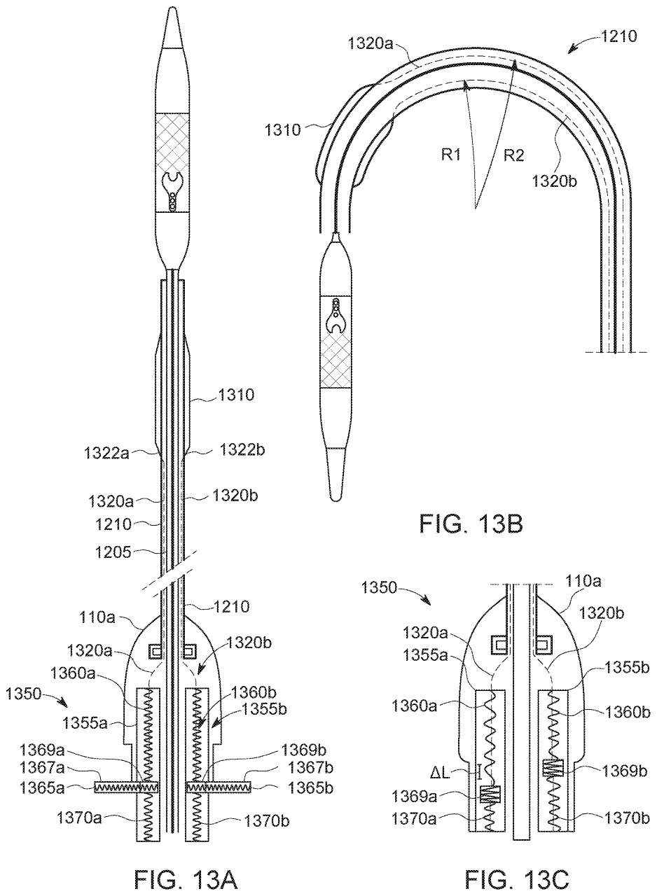

FIG. 13 illustrates a delivery system 1200 incorporating an anchoring system 1300 according to one or more aspects of the disclosure.

The anchoring system 1300 can include an anchor 1310 and one or more control wires 1320 coupled to the anchor 1310. The anchor 1310 can be any shape, and in one example is a cone or frustocone or a curved cone or curved frustocone. In the example of a curved cone or curved frustocone, the anchor 1310 can define a first end 1312 (e.g., an outflow end), a second end 1314 (e.g., an inflow end), and a curved surface 1316 between the first end 1312 and the second end 1314. An interior surface of the curved surface 1316 can define an opening or lumen 1318 extending between the first end 1312 and the second end 1314. As shown, a portion of the delivery system 1200, such as the delivery catheter 1210, may pass through the opening or lumen 1318 defined by the anchor 1310.

The anchor 1310 can be made of any material, such as a shape memory material, including polymers or metals or metal alloys, including nickel-titanium alloys (e.g., Nitinol). The anchor 1310 can be, for example, laser cut from a tube of the shape memory material, or formed by braiding strands of shape memory material into a mesh configuration, allowing for bloodflow through cells or apertures defined by the mesh. In this regard, the anchor 1310 can be shape set, for example via heat treatment, to the shape shown in FIG. 13 (or other similar shapes described herein) so that it tends to revert to that shape in the absence of applied forces. The set shape may generally correspond to an expanded or deployed configuration of the anchor 1310, as shown in FIG. 13. While FIG. 13 depicts a mesh configuration defining cells or apertures, the anchor 1310 can have any type of configuration that defines cells or apertures of sufficient to size to allow bloodflow through and past the anchor 1310.

In the expanded or deployed configuration, the first end 1312 can have a cross-sectional diameter that is greater than a cross-sectional diameter of the second end 1314, and the curved surface 1316 can taper outwardly from the second end 1314 to the first end 1312 such that a cross-sectional diameter of the curved surface 1316 generally increases from the second end 1314 to the first end 1312. The lumen 1318, similar to the curved surface 1316, can have a cross-sectional diameter that increases from the second end 1314 to the first end 1312. To allow one or more components of the delivery system 1200 to pass through the lumen 1318 of the anchor 1310, the lumen 1318 can have a minimum cross-sectional diameter, for example at the second end 1314, that is greater than or equal to an outer diameter of one or more components of the delivery system 1200, for example the delivery catheter 1210. The second end 1314 of the anchor 1310 may be coupled to the delivery catheter 1210 via any suitable modality, including for example via adhesives, via suturing, via welding, or the like.

As described above, the anchoring system 1300 can include one or more control wires 1320. The control wires 1320 may have any suitable form that permits transition of tension, including for example sutures. A first (or distal or leading) end 1322 of at least one, at least some, or all of the control wires 1320 can be coupled to the anchor 1310. In one example, the one or more control wires 1320 are coupled to the first end 1322 of the anchor. In another example, the one or more control wires 1320 are coupled to the curved surface 1316. In this example, the one or more control wires 1320 can be coupled near the first end 1312 (e.g., closer to the first end 1312 than the second end 1314). A second (or proximal or trailing) end of at least one, at least some, or all of the control wires 1320 can be coupled to one or more components of the delivery system. For example, the second end of the control wire(s) 1320 can be coupled to a handle (e.g., handle 110) such that the tension can be provided to the one or more control wires 1320 by one or more control features on the handle. Any configuration in which the control wire 1320 can be tensioned by the user (including via manual tension) may be suitable.

FIGS. 13A-B illustrate the anchoring system 1300 of FIG. 13 incorporating an exemplary control wires 1320a-b configuration and tensioning mechanism 1350 according to one or more aspects of the disclosure. As shown, the control wires 1320a-b can extend distally from the handle 110a, through respective control wire lumens 1305 inside a wall of the delivery catheter 1210. At a point of the delivery catheter 1210 near the anchor 1310, the control wires 1320a-b can exit the control wire lumens 1305 to extend outward from the delivery catheter 1210, such as by a hole, so that the first ends 1322a-b can couple to the anchor 1310 (shown in the compressed or stowed configuration).

FIG. 13B depicts a portion of the anchoring system 1300 of FIG. 13A incorporating the exemplary control wires 1320a-b configuration according to one or more aspects of the disclosure. As shown, the delivery catheter 1210 is in a bent or curved configuration, which can occur while the delivery catheter 1210 is being delivered through the aortic arch AA (as shown in FIGS. 12 and 13 above). By virtue of this curved configuration, the control wires 1320a-b bend or curve according to different radiuses of curvature, with control wire 1320a having a radius of curvature R₂ that is greater than a radius of curvature R₁ of control wire 1320b. The length of control wire 1320a is proportional to π*R₂ and the length of control wire 1320b is proportional to π*R₁ and a difference in the lengths can be ΔL. To account for the difference in length ΔL and to ensure uniform tensioning between control wires 1320a-b, the handle 110a can incorporate a tensioning mechanism 1350 as shown in FIGS. 13A-B.

The tensioning mechanism 1350 can include a pair of housings 1355a-b (which may be cavities or recesses) that can be fixed relative to handle 110a. Housing 1355a can have a first spring 1360a and a second spring 1370a disposed therein. Similarly, housing 1355b can have a first spring 1360b and a second spring 1370b disposed therein. A distal end of first spring 1360a can be coupled to (or otherwise abut without a fixed coupling) an internal surface of housing 1355a and a proximal end of first spring 1360a can be coupled to an anchor 1369a of spring depressor 1365a. A distal end of spring 1370a can be coupled to the anchor 1369a of spring depressor 1365a and a proximal end of spring 1370a can be coupled to (or otherwise abut without a fixed coupling) an internal surface of housing 1355a. The spring depressor 1365a can have a handle 1367a capable of being gripped, pushed, and/or pulled by a user. The control wire 1320a can be coupled to the anchor 1369a and can pass through a central bore generally defined by first spring 1360a.

Similarly, a distal end of first spring 1360b can be coupled to (or otherwise abut without a fixed coupling) an internal surface of housing 1355b and a proximal end of first spring 1360b can be coupled to an anchor 1369b of spring depressor 1365b. A distal end of spring 1370b can be coupled to the anchor 1369b of spring depressor 1365b and a proximal end of spring 1370b can be coupled to (or otherwise abut without a fixed coupling) an internal surface of housing 1355b. The spring depressor 1365b can have a handle 1367b capable of being gripped, pushed, and/or pulled by a user. The control wire 1320b can be coupled to the anchor 1369b and can pass through a central bore generally defined by first spring 1360b.

Tension can be applied or released from control wires 1320a-b by advancing or withdrawing spring depressors 1365a-b relative to handle 110a. For example, to increase tension on control wires 1320a-b, the spring depressors 1365a-b can be withdrawn proximally so that anchors 1369a-b translate proximally relative to housings 1355a-b. In the example of FIG. 13, increasing tension on control wires 1320a-b causes the anchor 1310 to move from the expanded or deployed configuration of FIG. 13 to the collapsed or stowed configuration. To prevent premature deployment or expansion of anchor 1310 during delivery, the springs 1360a-b can have a spring constant k that is great enough to overcome the self-expansion force exerted by anchor 1310 when it is in the collapsed or stowed condition, but not so great as to prevent hinder expansion of the anchor 1310 via spring depressors 1365a. In other examples, the spring constant k may not be enough to overcome the self-expansion force exerted by anchor 1310 when it is in the collapsed or stowed condition. In this regard, a user can manually hold the spring depressors 1365a-b in place to avoid premature deployment or expansion of anchor 1310. In other exemplary anchoring systems (e.g., anchoring system 1600), increasing tension on control wires 1320a-b may cause the anchor to move from the collapsed or stowed configuration to the expanded or deployed configuration. Proximal translation of the anchors 1369a-b exerts tension on control wires 1320a-b. To release tension, the spring depressors 1365a-b can be advanced distally so that anchors 1369a-b translate distally relative to housings 1355a-b.

To account for the length difference ΔL, each of the springs 1360a-b and 1370a-b can have an uncompressed length that is greater than ΔL so that during the application or release of tension, the anchors 1369a-b do not advance too far proximally or distally relative to housings 1355a-b so as to confront the interior surface of housings 1355a-b.

FIG. 13C depicts the tensioning mechanism 1350 depicting anchors 1369a-b of spring depressors 1365a-b but with handles 1367a-b omitted for clarity. As shown, the anchor 1369a is offset proximally relative to anchor 1369b by the length difference ΔL to ensure uniform tension to both control wires 1320a-b. The tensioning mechanism 1350 described above can be incorporated into any anchoring mechanism described in the present application in order to apply tension to control wires and/or deploy an anchor, such as anchoring systems 1300, 1400, 1600, and/or 1700.

Returning back to FIG. 13, upon application of tension to the control wires 1320, a radially inward force can be applied to first end 1312. This radially inward force is sufficient to compress the anchor 1310 from the shape-set expanded or deployed configuration to a compressed or stowed configuration.

In the compressed or stowed configuration, the anchor 1310 can be substantially cylindrical such that a cross-sectional diameter of the first end 1312 is approximately the same as the cross-sectional diameter of the second end 1314 and the lumen 1318 has a substantially constant cross-sectional diameter between the first end 1312 and the second end 1314. In the compressed or stowed configuration, the cross-sectional diameter of the anchor 1310 can be equal or lesser than a cross-sectional diameter of the prosthetic heart valve 1250 in the crimped or collapsed configuration and/or a cross-sectional diameter of the balloon 1220 when deflated so that the anchor 1310 does not interfere with patient anatomy during delivery. However, in other embodiments, that cross-sectional diameter of the anchor 1310 in the stowed condition may be larger than the crimped diameter of the prosthetic heart valve 1250 without causing interference with the patient's anatomy during delivery.

In operation, the delivery catheter 1210 may be advanced until the prosthetic heart valve 1250, while still crimped or collapsed, is positioned within the native aortic valve annulus A. In one example, delivery catheter 1210 may be advanced until the inflow edge of the prosthetic heart valve 1250 is positioned between about 0 mm and about 8 mm subannularly with respect to the basal plane of the native aortic valve annulus A.

During advancement, the anchor 1310 of the anchoring system 1300 can be in the stowed or compressed position. The anchor 1310 may be held in the stowed or compressed position by application of tension on control wires 1320, pulling the second end 1312 firmly against an exterior of delivery catheter 1210. While in the stowed condition, the cross-sectional diameter of the anchor 1310 is preferably smaller than the interior diameter of the vasculature, including the aortic arch AA, so that the anchor 1310 does not excessively interfere with the patient's anatomy during advancement of the delivery catheter 1210.

After the prosthetic heart valve 1250 is in the desired positioned relative to the native aortic valve annulus A, and before expansion of the balloon 1220 begins, the anchoring system 1300 may be deployed or expanded to engage with an interior surface of the aortic arch AA. Where the anchor 1310 is a cone (or frustocone) or curved cone (or curved frustocone), such as described above with respect to FIG. 13, some or all of the tension on control wires 1320 can be released (e.g., by handle) to allow the anchor 1310 to expand radially outwardly from the delivery catheter 1210 and toward the interior wall of the aorta. The first end 1312 of the anchor 1310 can expand until it contacts an interior surface of the aortic arch AA (and/or the ascending aorta) and exerts a slight force on the interior surface of the aortic arch AA (and/or the ascending aorta), thus resisting axial movement of the delivery system 1200. This exerted force serves to anchor the delivery system 1200 relative to the aortic arch AA (and/or the ascending aorta, and thus helps to stabilize the axial position of the prosthetic heart valve 1250 relative to the basal plane of the native aortic valve annulus A during subsequent inflation of the balloon 1220 and deployment of the prosthetic heart valve 1250. In the example where the anchor 1310 is formed as a braided mesh, the anchor 1310 can also provide embolic protection. For example, any material (such as plaque or calcium) that embolizes during deployment of the prosthetic heart valve 1250 may become trapped within the anchor 1310 before moving downstream through the vasculature where the debris may cause stroke or other infarctions. In this respect, when deploying the anchor 1310, it may be preferable in some examples for the first end 1312 to be positioned upstream of the brachiocephalic artery.

Once anchored, the balloon 1220 can be expanded to expand and implant the prosthetic heart valve 1250. Since the delivery system 1200 is anchored relative to the aortic arch AA, axial movement of the prosthetic heart valve 1250 via axial movement of the delivery catheter 1210 and/or axial movement of the balloon 1220 is limited or prevented, ensuring proper placement of the prosthetic heart valve 1250. Since the delivery device 1200 has been anchored, it may not be necessary to finely adjust the axial positioning of the partially-expanded prosthetic heart valve relative to the native aortic valve annulus A (as described above in step 214). However, it may still be preferable to perform fine axial adjustment to account for any limited axial foreshortening of the frame (e.g., frame 20 described above) of the prosthetic heart valve 1250 itself as the prosthetic heart valve 1250 radially expands.

After deployment of the prosthetic heart valve 1250 is complete, tension can again be applied to control wires 1320, compressing the anchor 1310 from the shape-set expanded or deployed configuration to the compressed or stowed configuration and allowing the anchor 1310 to be released from the patient anatomy and allowing the delivery catheter 1310 to move axially. The balloon 1220 may then be deflated (although the balloon 1220 may be deflated prior to stowing the anchor 1310) and the delivery catheter 1210 and guidewire may be removed from the patient to complete the procedure.

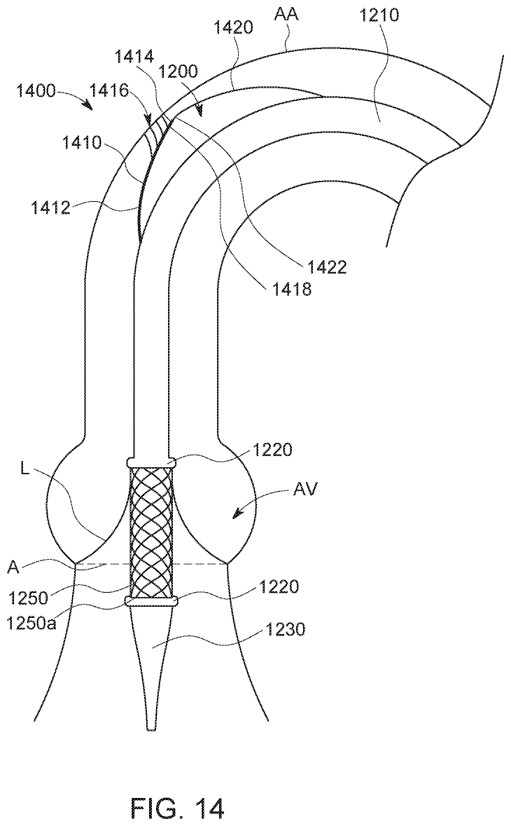

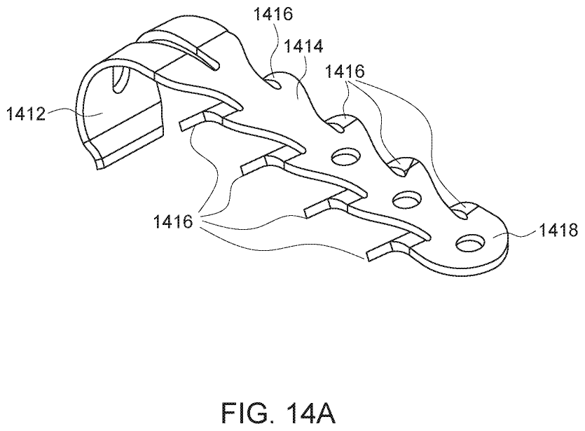

FIG. 14 illustrates a delivery system 1200 incorporating an anchoring system 1400 according to one or more aspects of the disclosure.

The anchoring system 1400 can include an anchor 1410 and one or more control wires 1420 coupled to the anchor 1410. The anchor 1410 can have a base section 1412 and a gripping section 1414. The gripping section 1414 can include one or more frictional elements 1416 extending from the gripping section 1414. An exemplary anchor 1410 can be seen in FIG. 14A.

As shown in FIG. 14A, the frictional elements 1416 can be arranged along a length of the gripping section 1414 and can be arranged in one or more columns and one or more rows. Frictional elements 1416 may comprise frictional protrusions or tines having tapering pointed tips extending from a side of gripping section 1414. Frictional elements 1416 may extend at a right angle relative to gripping section 1414. In another example, frictional elements 1416 may be angled relative to the gripping section 1414. In this regard, the tips of the frictional elements 1416 can be angled away from base section 1412 (e.g., point in an antegrade direction), which may help prevent frictional elements 1414 from inadvertently snaring tissue during delivery. It may be appreciated that any suitable frictional elements may be used, such as prongs, windings, bands, barbs, grooves, channels, bumps, surface roughening, sintering, high-friction pads, coverings, coatings, or a combination of these. However, it should be noted that some types of frictional elements that can be utilized may permanently alter or cause some trauma to the tissue engaged. Thus, it is preferable that frictional elements 1416 be atraumatic and generally frictional rather than penetrative so as to not injure or otherwise affect the tissue in a clinically significant way.

The anchor 1410 can be made of any material, such as a shape memory materials, including polymers, metals, or metal alloys, including nickel-titanium alloys (e.g., Nitinol). The anchor 1410 can be laser cut from a sheet of shape memory material. In this regard, the anchor 1410 can be shape set, for example via heat setting, so that it tends to revert to the set shape in the absence of applied forces. The set shape may be similar or identical to the shape achieved in an expanded or deployed configuration of the anchor 1410, as shown in FIG. 14.

The base section 1412 can be coupled to the delivery catheter 1210 in any suitable fashion, such as be adhesives, via suturing, via welding or the like, such that the frictional elements 1416 face radially outward from the delivery catheter 1210 or the balloon 1220 and toward an internal wall of the aorta (either at a right angle from gripping section 1414 or in an angled manner, as described above) in order to frictionally engage the tissue when the anchor 1410 is deployed, without having a high risk of frictional engagement with tissue when the anchor 1410 is stowed or compressed.

As described above, the anchoring system 1400 can include one or more control wires 1420, which may be sutures or any other element capable of transmitting tension to the anchor 1410 when being pulled by a user. A first (or distal or leading) end 1422 of at least one, at least some, or all of the control wires 1420 can be coupled to the anchor 1410. In one example, the one or more control wires 1420 are coupled to the proximal end 1418 of the gripping section 1414. In another example, the one or more control wires 1420 are coupled to the gripping section 1414 near the proximal end 1418 (e.g., closer to the proximal end 1418 than base section 1412). A second (or proximal or trailing) end of at least one, at least some, or all of the control wires 1420 can be coupled to one or more components of the delivery system. For example, the second end of the control wire(s) 1420 can be coupled to a handle (e.g., handle 110) such that the tension can be provided to the one or more control wires 1320 by one or more control features on the handle. In this regard, the control wire(s) 1420 can extend distally from the handle, through a control wire lumen inside a wall of the delivery catheter 1210. At a point of the delivery catheter 1210 near the anchor 1410, the control wire(s) 1420 can extend exit the control wire lumen outward from the delivery catheter 1210, such as by a hole, so that the first end 1422 can couple to the anchor 1410. However, any configuration in which the control wire 1420 can be tensioned by the user (including via manual tension) may be suitable.

Upon application of tension to the control wire(s) 1420, a radially inward force can be applied to proximal end 1418. This radially inward force is sufficient to compress the anchor 1410 from the shape-set expanded or deployed configuration to a compressed or stowed configuration.

In operation, the delivery catheter 1210 may be advanced until the prosthetic heart valve 1250, while still crimped or collapsed, is positioned within the native aortic valve annulus A. In one example, delivery catheter 1210 may be advanced until the inflow edge of the prosthetic heart valve 1250 is positioned between about 0 mm and about 8 mm subannularly with respect to the basal plane of the native aortic valve annulus A.

During advancement, the anchor 1410 of the anchoring system 1400 can be in the stowed or compressed position. The anchor 1410 is held in the stowed or compressed position by application of tension on control wires 1420, pulling the second end 1312 firmly against an exterior of delivery catheter 1210. Since anchor 1410, in the stowed or compressed position, extends radially outward from the central axis of the delivery catheter 1210 a smaller distance than the distance which the prosthetic heart valve 1250 (in the crimped or collapsed configuration) and/or balloon 1220 extends radially outwardly from the central axis of the delivery catheter 1210, the anchor 1410 does not excessively interfere with the patient's anatomy during advancement of the delivery catheter 1210.

Optionally, a channel, groove, depression, or indentation may be formed in an outer wall of the delivery catheter 1210, allowing the anchor 1410 to fit within an atraumatic profile defined by the delivery catheter 1210 so that the anchor does not interfere with the patient's anatomy during advancement of the delivery catheter 1210. In other words, in the stowed condition of the anchor 1410, little or no structure of the anchor 1410 may extend radially outwardly form the outermost surface of the delivery catheter 1210 if the anchor 1410 is received within the channel or groove while stowed.

After the prosthetic heart valve 1250 is in the desired positioned relative to the native aortic valve annulus A, and before expansion of the balloon 1220 begins, the anchoring system 1300 may be deployed or expanded to engage with an interior surface of the aortic arch AA. Where the anchor 1410 includes a gripping section 1414 and frictional elements 1416, some or all of the tension on the control wires 1420 can be released (e.g., by handle) to allow the anchor 1410 to expand radially outwardly from the delivery catheter 1210 and toward the interior wall of the aorta. At least the gripping section 1414 of the anchor 1410 can expand until it contacts an interior surface of the aortic arch AA (and/or the ascending aorta) and exerts a slight force on the interior surface of the aortic arch AA (and/or the ascending aorta), resulting in frictional engagement of the frictional elements 1416 with the aortic arch AA (and/or the ascending aorta)and thus resisting axial movement of the delivery system 1200. This frictional engagement serves to anchor the delivery system 1200 relative to the aortic arch AA (and/or the ascending aorta, and thus helps to stabilize the axial position of the prosthetic heart valve 1250 relative to the basal plane of the native aortic valve annulus A during subsequent inflation of the balloon 1220 and deployment of the prosthetic heart valve 1250.

Once anchored, the balloon 1220 can be expanded to expand and implant the prosthetic heart valve 1250. Since the delivery system 1200 is anchored relative to the aortic arch AA, axial movement of the prosthetic heart valve 1250 via axial movement of the delivery catheter 1210 and/or axial movement of the balloon 1220 is limited or prevented, ensuring proper placement of the prosthetic heart valve 1250. Since the delivery device 1200 has been anchored, it may not be necessary to finely adjust the axial positioning of the partially-expanded prosthetic heart valve relative to the native aortic valve annulus A (as described above in step 214). However, it may still be preferable to perform fine axial adjustment to account for any limited axial foreshortening of the frame (e.g., frame 20 described above) of the prosthetic heart valve 1250 itself as the prosthetic heart valve 1250 radially expands.

After deployment of the prosthetic heart valve 1250 is complete, tension can again be re-applied from the control wires 1420, compressing the anchor 1410 from the shape-set expanded or deployed configuration to the compressed or stowed configuration and allowing the anchor 1410 to be released from the patient anatomy and allowing the delivery catheter 1310 to move axially. The balloon 1220 may then be deflated (although the balloon 1220 may be deflated prior to stowing the anchor 1310) and the delivery catheter 1210 and guidewire may be removed from the patient to complete the procedure.

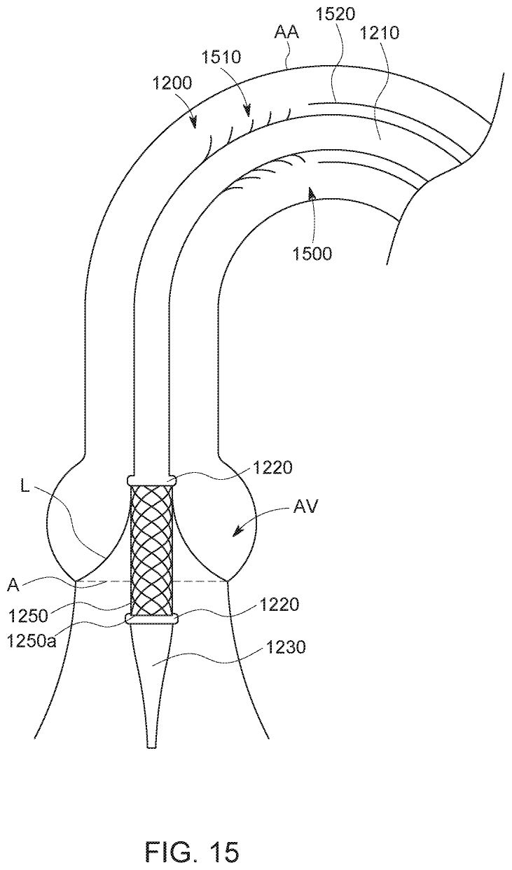

FIG. 15 illustrates a delivery system 1200 incorporating an anchoring system 1500 according to one or more aspects of the disclosure.

The anchoring system 1500 can include an anchor 1510 and a sheath 1520. The anchor 1510 can include a textured section on an external surface of delivery catheter 1210. The textured section can be formed by any suitable technique. In one example, the textured section can be formed by material settling or slitting the delivery catheter 1210 to create the texture and then reflowing in short segments to the delivery catheter 1210. The textured section can be biased in a predetermined direction, and in one example is biased in the antegrade direction to allow for frictional engagement with the aortic wall to reduce or limit antegrade movement of the delivery catheter 1210 during balloon expansion.

A retractable sheath 1520 can be positioned over and radially outward of the anchor 1510 to prevent engagement of the anchor 1510 with the patient anatomy during delivery. The sheath 1520 can have a diameter that is greater than a diameter of the delivery catheter 1210, such that the sheath 1520 can be advanced over the delivery catheter 1210, in particular the anchor 1310 comprising the textured section, to cover the textured section, thereby preventing the textured section from engaging with patient anatomy, and retracted to expose the anchor 1510 to allow for the frictional engagement. The sheath 1520 can optionally have a cross-sectional diameter that is equal to or less than a cross-sectional diameter of the prosthetic heart valve 1250 in the crimped or collapsed configuration.

In operation, the delivery catheter 1210 may be advanced until the prosthetic heart valve 1250, while still crimped or collapsed, is positioned within the native aortic valve annulus A. In one example, delivery catheter 1210 may be advanced until the inflow edge of the prosthetic heart valve 1250 is positioned between about 0 mm and about 8 mm subannularly with respect to the basal plane of the native aortic valve annulus A.

During advancement, the anchor 1510 of the anchoring system 1500 can be partially or completely stowed by virtue of being partially or completely covered by the sheath 1520. While in the stowed condition, the cross-sectional diameter of the sheath 1520 is preferably smaller than the interior diameter of the vasculature, including the aortic arch AA, so that the anchor 1520 does not excessively interfere with the patient's anatomy during advancement of the delivery catheter 1210.

After the prosthetic heart valve 1250 is in the positioned relative to the native aortic valve annulus A, and before expansion of the balloon 1220 begins, the anchoring system 1500 may be deployed to engage with an interior surface of the aortic arch AA. In this regard, the sheath 1520 can be retracted, exposing at least some or all of the anchor 1510. When the anchor 1510 includes a textured section, the anchor 1510, when exposed by sheath 1520, contacts an interior surface of the aortic arch AA (and/or the ascending aorta) and exerts a slight force on the interior surface of the aortic arch AA (and/or the ascending aorta), resulting in frictional engagement of the frictional elements anchor 1410 with the aortic arch AA (and/or the ascending aorta) and thus resisting axial movement of the delivery system 1200.. This frictional engagement serves to anchor the delivery system 1200 relative to the aortic arch AA (and/or the ascending aorta, and thus helps to stabilize the axial position of the prosthetic heart valve 1250 relative to the basal plane of the native aortic valve annulus A during subsequent inflation of the balloon 1220 and deployment of the prosthetic heart valve 1250.

Once anchored, the balloon 1220 can be expanded to expand and implant the prosthetic heart valve 1250. Since the delivery system 1200 is anchored relative to the aortic arch AA, axial movement of the prosthetic heart valve 1250 via axial movement of the delivery catheter 1210 and/or axial movement of the balloon 1220 is limited or prevented, ensuring proper placement of the prosthetic heart valve 1250. Since the delivery device 1200 has been anchored, it may not be necessary to finely adjust the axial positioning of the partially-expanded prosthetic heart valve relative to the native aortic valve annulus A (as described above in step 214). However, it may still be preferable to perform fine axial adjustment to account for any limited axial foreshortening of the frame (e.g., frame 20 described above) of the prosthetic heart valve 1250 itself as the prosthetic heart valve 1250 radially expands.

After deployment of the prosthetic heart valve 1250 is complete, the anchor 1510 can be returned to the stowed position by advancing the sheath 1520 over the anchor 1510 and allowing the anchor 1510 to be released from the patient anatomy and allowing the delivery catheter 1310 to move axially. The balloon 1220 may then be deflated (although the balloon 1220 may be deflated prior to stowing the anchor 1310) and the delivery catheter 1210 and guidewire may be removed from the patient to complete the procedure.

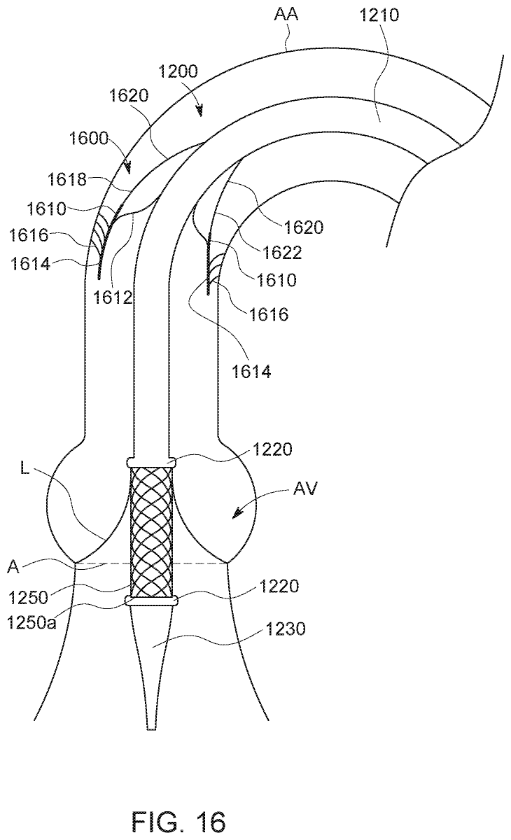

FIG. 16 illustrates a delivery system 1200 incorporating an anchoring system 1600 according to one or more aspects of the disclosure.

The anchoring system 1600 can include at least one anchor 1610 and one or more control wires 1620 coupled to the at least one anchor 1610. In the example of FIG. 16, the anchoring system 1600 includes three anchors 1610, with each of the three anchors 1610 having a corresponding control wire 1620. In this regard, each of the three anchors 1610 can be regularly-spaced to provide a centering effect and to provide equal balance against the aortic wall. In other examples, any number of anchors 1610 and/or control wires 1620 can be implemented. Each anchor 1610 can have a base section 1612 and a gripping section 1614. The gripping section 1614 can include one or more frictional elements 1616 extending from the gripping section 1614.

The anchor 1610 can be made of any material, such as a shape memory materials, including polymers, metals, or metal alloys, including nickel-titanium alloys (e.g., Nitinol). The anchor 1610 can be laser cut from a sheet of shape memory material. In this regard, the anchor 1610 can be shape set, for example via heat setting, so that it tends to revert to the set shape in the absence of applied forces. The set shape may be in a stowed or compression configuration in which the anchor 1610 is compressed against delivery catheter 1210.

As shown in FIG. 16, the frictional elements 1616 can be arranged along a length of the gripping section 1614 and can be arranged in one or more columns and one or more rows. Frictional elements 1616 may comprise frictional protrusions or tines having tapering pointed tips extending from a side of gripping section 1614. Frictional elements 1616 may extend at a right angle relative to gripping section 1614. In another example, frictional elements 1616 may be angled relative to the gripping section 1614. In this regard, the tips of the frictional elements 1616 can be angled away from base section 1612 (e.g., point in an antegrade direction), which may help prevent frictional elements 1614 from inadvertently snaring tissue during delivery. It may be appreciated that any suitable frictional elements may be used, such as prongs, windings, bands, barbs, grooves, channels, bumps, surface roughening, sintering, high-friction pads, coverings, coatings, or a combination of these. However, it should be noted that some types of frictional elements that can be utilized may permanently alter or cause some trauma to the tissue engaged. Thus, it is preferable that frictional elements 1616 be atraumatic and generally frictional rather than penetrative so as to not injure or otherwise affect the tissue in a clinically significant way.

The base section 1612 can be coupled to the delivery catheter 1210 in any suitable fashion, such as be adhesives, via suturing, via welding or the like, such that the frictional elements 1616 face radially outward from the delivery catheter 1210 or the balloon 1220 and toward an internal wall of the aorta (either at a right angle from gripping section 1614 or in an angled manner, as described above) in order to frictionally engage the tissue when the anchor 1610 is deployed, without having a high risk of frictional engagement with tissue when the anchor 1610 is stowed or compressed.

As described above, the anchoring system 1600 can include one or more control wires 1620, which may be sutures or any other element capable of transmitting tension to the anchor 1610 when being pulled by a user. A first (or distal or leading) end 1622 of at least one, at least some, or all of the control wires 1620 can be coupled to the anchor 1610. In one example, the one or more control wires 1620 are coupled to the proximal end 1618 of the gripping section 1614. In another example, the one or more control wires 1620 are coupled to the gripping section 1614 near the proximal end 1618 (e.g., closer to the proximal end 1618 than a distal portion of gripping section 1614). A second (or proximal or trailing) end of at least one, at least some, or all of the control wires 1620 can be coupled to one or more components of the delivery system. For example, the second end of the control wire(s) 1620 can be coupled to a handle (e.g., handle 110) such that the tension can be provided to the one or more control wires 1620 by one or more control features on the handle. In this regard, the control wire(s) 1620 can extend distally from the handle, through a control wire lumen inside a wall of the delivery catheter 1210. At a point of the delivery catheter 1210 near the anchor 1610, the control wire(s) 1620 can extend exit the control wire lumen outward from the delivery catheter 1210, such as by a hole, so that the first end 1622 can couple to the anchor 1610. However, any configuration in which the control wire 1620 can be tensioned by the user (including via manual tension) may be suitable.

Upon application of tension to the sutures 1620, a radially outward force can be applied to proximal end 1618. This radially outward force is sufficient to expand the anchor 1610 from the shape-set compressed or stowed configuration to an expanded or deployed configuration. In one example, a predetermined amount of spring tension is applied to the sutures so as not to exceed a predetermined amount of force exerted on the patient anatomy.

In operation, the delivery catheter 1210 may be advanced until the prosthetic heart valve 1250, while still crimped or collapsed, is positioned within the native aortic valve annulus A. In one example, delivery catheter 1210 may be advanced until the inflow edge of the prosthetic heart valve 1250 is positioned between about 0 mm and about 8 mm subannularly with respect to the basal plane of the native aortic valve annulus A.

During advancement, the anchor 1610 of the anchoring system 1600 can be in the shape-set stowed or compressed position, holding the gripping section 1614 and frictional elements 1616 of anchor 1610 toward the delivery catheter 1210. Since the anchor 1610 is shape set in the stowed or compressed configuration, tension on control wires 1620 is not required to maintain the stowed or compression configuration. Optionally, a retractable sheath (e.g. similar to sheath 1520) can be positioned over the anchor 1610. Since anchor 1610, or the optional retractable sheath, in the stowed or compressed position, extends radially outward from the central axis of the delivery catheter 1210 a smaller distance than the distance which the prosthetic heart valve 1250 (in the crimped or collapsed configuration) and/or balloon 1220 extends radially outwardly from the central axis of the delivery catheter 1210, the anchor 1610 (or the optional retractable sheath) does not excessively interfere with the patient's anatomy during advancement of the delivery catheter 1210.

After the prosthetic heart valve 1250 is in the desired positioned relative to the native aortic valve annulus A, and before expansion of the balloon 1220 begins, the anchoring system 1300 may be deployed or expanded to engage with an interior surface of the aortic arch AA. Where the anchor includes a gripping section 1614 and frictional elements 1616, tension can be applied (e.g., by handle) to the control wires 1620 that is sufficient to expand the anchor 1610, forcing the anchor 1610 to expand outward from the delivery catheter 1210 and toward the aortic wall. At least the gripping section 1614 of the anchor 1610 can expand until it contacts an interior surface of the aortic arch AA (and/or the ascending aorta) and exerts a slight force on the interior surface of the aortic arch AA (and/or the ascending aorta), resulting in frictional engagement of the frictional elements 1616 with the aortic arch AA (and/or the ascending aorta) and thus resisting axial movement of the delivery system 1200. This frictional engagement serves to anchor the delivery system 1200 relative to the aortic arch AA (and/or the ascending aorta, and thus helps to stabilize the axial position of the prosthetic heart valve 1250 relative to the basal plane of the native aortic valve annulus A during subsequent inflation of the balloon 1220 and deployment of the prosthetic heart valve 1250. Since the anchor 1610 is shape set in the stowed or compressed configuration, tension on control wires 1620 is required to maintain the expanded or deployed configuration during delivery. Optionally, the handle can include a locking mechanism to lock the control wire in a desired tension state to maintain the expanded or deployed configuration during delivery

Once anchored, the balloon 1220 can be expanded to expand and implant the prosthetic heart valve 1250. Since the anchor 1610 is shape set in the stowed or compressed configuration, tension on control wires 1620 is required to maintain the expanded or deployed configuration during expansion of the balloon 1220 and/or prosthetic heart valve 1250. Since the delivery system 1200 is anchored relative to the aortic arch AA, axial movement of the prosthetic heart valve 1250 via axial movement of the delivery catheter 1210 and/or axial movement of the balloon 1220 is limited or prevented, ensuring proper placement of the prosthetic heart valve 1250. Since the delivery device 1200 has been anchored, it may not be necessary to finely adjust the axial positioning of the partially-expanded prosthetic heart valve relative to the native aortic valve annulus A (as described above in step 214). However, it may still be preferable to perform fine axial adjustment to account for any limited axial foreshortening of the frame (e.g., frame 20 described above) of the prosthetic heart valve 1250 itself as the prosthetic heart valve 1250 radially expands. Advantageously, where anchoring system 1600 includes three anchors, each of the three anchors 1610 can be regularly-spaced to provide a centering effect and to provide equal balance against the aortic wall. This centers the prosthetic heart valve 1250 within the native aortic valve annulus A so that the prosthetic heart valve 1250 comes into contact with the native aortic valve annulus A at generally the same time around the circumference, further ensuring proper positioning of the prosthetic heart valve 1250.

After deployment of the prosthetic heart valve 1250 is complete, some or all of the tension can be released from the control wires 1620. Since the anchor 1610 is shape set in the stowed or compressed configuration, releasing the tension on control wires 1620 reduces or eliminates the applied forces to the anchors 1610, allowing the anchors 1610 return to the shape set compressed or stowed configuration and allowing the anchor 1610 to be released from the patient anatomy and allowing the delivery catheter 1310 to move axially. The balloon 1220 may then be deflated (although the balloon 1220 may be deflated prior to stowing the anchor 1310) and the delivery catheter 1210 and guidewire may be removed from the patient to complete the procedure.

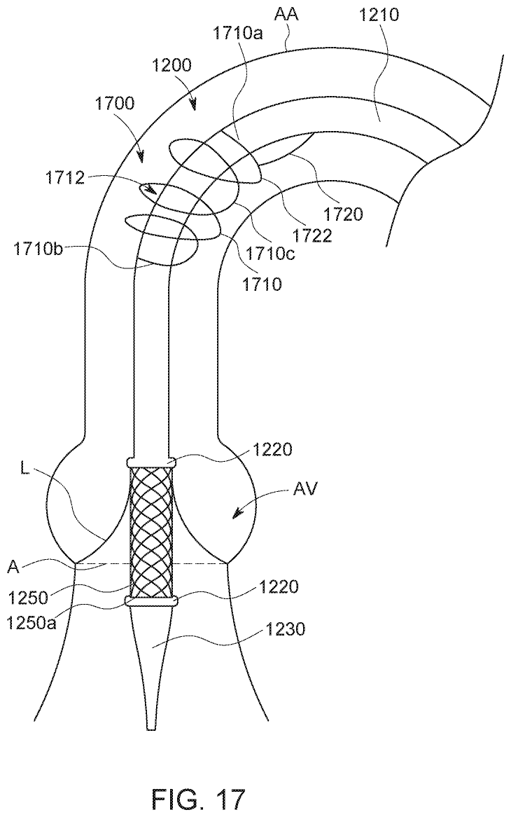

FIG. 17 illustrates a delivery system 1200 incorporating an anchoring system 1700 according to one or more aspects of the disclosure.

The anchoring system 1700 can include an anchor 1710 and one or more control wires 1720 coupled to the anchor 1710. The anchor 1710 can be any shape, and in one example is a single helix structure formed of a single or multi-filament wire defining an opening or lumen 1712. In the example of a single helix structure, the anchor 1710 can define a first end 1710a (e.g., an outflow end), a second end 1710b (e.g., an inflow end), and a coiled section 1710c of wire extending therebetween in a helical manner. An interior surface of the coiled section 1710c can define the opening or lumen 1718 extending between the first end 1710a and the second end 1710b. As shown, a portion of the delivery system 1200, such as the delivery catheter 1210, may pass through the opening or lumen 1718 defined by the anchor 1710.

The anchor 1710 can be made of any material, such as a shape memory material, including polymers or metals or metal alloys, including nickel-titanium alloys (e.g., Nitinol). The anchor 1710 can be, for example, laser cut from a tube of the shape memory material, or formed by braiding strands of shape memory material into a mesh configuration. In this regard, the anchor 1710 can be shape set, for example via heat treatment, to the shape shown in FIG. 17 (or other similar shapes described herein) so that it tends to revert to that shape in the absence of applied forces. The set shape may generally correspond to an expanded or deployed configuration of the anchor 1710, as shown in FIG. 17.

A first end 1710a of the anchor 1710 can be coupled to the delivery catheter 1210 and/or the second end 1710b of the anchor 1710 can be coupled to the delivery catheter 1210 via any suitable modality, including for example via adhesives, via suturing, via welding, or the like. In another example, the first end 1710a and the second end 1710b are not coupled to the delivery catheter 1210, but anchor 1710 is held in the compressed or stowed configuration and against delivery catheter 1210 by application of tension from suture 1720.

As described above, the anchoring system 1700 can include one or more control wires 1720. The control wires 1720may have any suitable form that permits transition of tension, including for example sutures. A first (or distal or leading) end 1722 of at least one, at least some, or all of the control wires 1720 can be coupled to the anchor 1720. In one example, the one or more control wires 1720 are coupled to the first end 1710a of the anchor. In another example, the one or more control wires 1720 are coupled to the coiled section 1710c. In this example, the one or more control wires 1720 can be coupled near the first end 1710a (e.g., closer to the first end 1710a than the second end 1710b). A second (or proximal or trailing) end of at least one, at least some, or all of the control wires 1720 can be coupled to one or more components of the delivery system. For example, the second end of the control wire(s) 1720 can be coupled to a handle (e.g., handle 110) such that the tension can be provided to the one or more control wires 1720 by one or more control features on the handle. In this regard, the control wire(s) 1720can extend distally from the handle, through a control wire lumen inside a wall of the delivery catheter 1210. At a point of the delivery catheter 1210 near the anchor 1710, the control wire(s) 1720can extend exit the control wire lumen outward from the delivery catheter 1210, such as by a hole, so that the first end 1722 can couple to the anchor 1710. However, any configuration in which the control wire 1720 can be tensioned by the user (including via manual tension) may be suitable.

Upon application of tension to the control wires 1720, a radially inward force can be applied to first end 1710 of anchor 1710. This radially inward force is sufficient to compress the anchor 1710 from the shape-set expanded or deployed configuration to a compressed or stowed configuration.

In the compressed or stowed configuration, the anchor 1710 can be substantially cylindrical such that a cross-sectional diameter of the first end 1710a is approximately the same as the cross-sectional diameter of the second end 1710b and the opening or lumen 1318 has a substantially constant cross-sectional diameter between the first end 1710a and the second end 1710b. In the compressed or stowed configuration, the cross-sectional diameter of the anchor 1710 can be equal or lesser than a cross-sectional diameter of the prosthetic heart valve 1250 in the crimped or collapsed configuration and/or a cross-sectional diameter of the balloon 1220 when deflated so that the anchor 1710 does not interfere with patient anatomy during delivery. However, in other embodiments, that cross-sectional diameter of the anchor 1710 in the stowed condition may be larger than the crimped diameter of the prosthetic heart valve 1250 without causing interference with the patient's anatomy during delivery.

During advancement, the anchor 1710 of the anchoring system 1700 can be in the stowed or compressed position. The anchor 1710 may be held in the stowed or compressed position by application of tension on control wires 1710, pulling the second end 1710a firmly against an exterior of delivery catheter 1210. While in the stowed condition, the cross-sectional diameter of the anchor 1710 is preferably smaller than the interior diameter of the vasculature, including the aortic arch AA, so that the anchor 1710 does not excessively interfere with the patient's anatomy during advancement of the delivery catheter 1210.

After the prosthetic heart valve 1250 is in the desired positioned relative to the native aortic valve annulus A, and before expansion of the balloon 1220 begins, the anchoring system 1300 may be deployed or expanded to engage with an interior surface of the aortic arch AA. When the anchor is a single helix structure, such as described above in FIG. 13, some or all of the tension on control wires 1720 can be released (e.g., by handle) to allow the anchor 1710 to expand radially outwardly from the delivery catheter 1210 and toward the interior wall of the aorta. The first end 1710a, second end 1710b, and/or the coiled section 1710c of the anchor 1710 can expand until it contacts an interior surface of the aortic arch AA (and/or the ascending aorta) and exerts a slight force on the interior surface of the aortic arch AA (and/or the ascending aorta), thus resisting axial movement of the delivery system 1200. This exerted force serves to anchor the delivery system 1200 relative to the aortic arch AA (and/or the ascending aorta, and thus helps to stabilize the axial position of the prosthetic heart valve 1250 relative to the basal plane of the native aortic valve annulus A during subsequent inflation of the balloon 1220 and deployment of the prosthetic heart valve 1250.

Once anchored, the balloon 1220 can be expanded to expand and implant the prosthetic heart valve 1250. Since the delivery system 1200 is anchored relative to the aortic arch AA, axial movement of the prosthetic heart valve 1250 via axial movement of the delivery catheter 1210 and/or axial movement of the balloon 1220 is limited or prevented, ensuring proper placement of the prosthetic heart valve 1250. Since the delivery device 1200 has been anchored, it may not be necessary to finely adjust the axial positioning of the partially-expanded prosthetic heart valve relative to the native aortic valve annulus A (as described above in step 214). However, it may still be preferable to perform fine axial adjustment to account for any limited axial foreshortening of the frame (e.g., frame 20 described above) of the prosthetic heart valve 1250 itself as the prosthetic heart valve 1250 radially expands.

After deployment of the prosthetic heart valve 1250 is complete, tension can again be applied to control wires 1320, compressing the anchor 1310 from the shape-set expanded or deployed configuration to the compressed or stowed configuration and allowing the anchor 1310 to be released from the patient anatomy and allowing the delivery catheter 1310 to move axially. The balloon 1220 may then be deflated (although the balloon 1220 may be deflated prior to stowing the anchor 1310) and the delivery catheter 1210 and guidewire may be removed from the patient to complete the procedure.

### Conclusion

Although the various embodiments above are presented as individual embodiments, it should be understood that features of certain embodiments may be combined with features of other embodiments. For example, multiple anchors 1310/1410/1510/1610/1710 may be provided on a single device. As one example, anchor 1310 may provide for both anchoring and embolic protection, which may be combined with anchor 1410 to provide enhanced anchoring. Further, although the anchor systems described herein may be particularly useful for balloon-expandable transcatheter prosthetic heart valve procedures, the anchor systems may be used for (i) other TAVR procedures, including self-expandable prosthetic heart valves, (ii) other transcatheter heart valve implantation procedures, such as mitral, tricuspid, or pulmonary heart valve replacements, whether self-expandable or balloon-expandable, or (iii) other structural heart implantation procedures, including for example transcatheter edge-to-edge repair of the mitral valve or tricuspid valve or implantation of expandable occluders such as left atrial appendage (“LAA”) occluder implantation.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve implantation system, comprising:
a collapsible and expandable prosthetic heart valve;
a delivery system for delivering and deploying the collapsible and expandable prosthetic heart valve, the delivery system including a delivery catheter and an inflatable balloon at a distal end portion of the delivery catheter; and
an anchoring system operably coupled to the delivery system, the anchoring system including an anchor having a compressed or stowed condition, and an expanded or deployed condition, the anchor being configured engage with patient anatomy in the expanded or deployed configuration of the anchor to anchor the delivery system relative to patient anatomy to restrict axial movement of the delivery catheter during inflation of the inflatable balloon.

2. The system of claim 1, wherein the anchoring system includes:
at least one control wire coupled to the anchor, wherein applying tension to the control wire causes the anchor to either (i) move from the compressed or stowed configuration to the expanded or deployed configuration, or (ii) move from the expanded or deployed configuration to the compressed or stowed configuration.

3. The system of claim 2, wherein the anchoring system includes:
a tensioning mechanism disposed in a handle of the delivery system, the tensioning mechanism includes a plurality of springs coupled to the at least one control wire so as to apply a uniform tension to the at least one control wire.

4. The system of claim 2, wherein the anchor has a frustoconical or curved frustoconical shape defining a first end and a second end, the anchor being shape-set to the expanded or deployed configuration such that the anchor has the frustoconical or curved frustoconical shape in the absence of applied forces and, wherein upon application of tension from the at least one control wire, the anchor transitions from the expanded or deployed condition to the compressed or stowed configuration.

5. The system of claim 4, wherein the first end of the anchor, in the expanded or deployed configuration, is sized and shaped to contact and exert a force on the patient anatomy to anchor the delivery system relative to the patient anatomy.

6. The system of claim 1, wherein the anchor is formed of a shape memory material, the shape memory material being a nickel-titanium alloy.

7. The system of claim 2, wherein the anchor includes a base section and a gripping section, the gripping section including a plurality of frictional elements, wherein the anchor is shape-set to the expanded or deployed configuration and, upon application of tension from the at least one control wire, the anchor compresses from the expanded or deployed configuration to the compressed or stowed configuration.

8. The system of claim 7, wherein the frictional elements, in the expanded or deployed configuration, are sized and shaped to frictionally engage with the patient anatomy to anchor the delivery system relative to the patient anatomy.

9. The system of claim 8, wherein the frictional elements are angled in an antegrade direction relative to the gripping section so as to prevent the frictional elements from inadvertently snaring tissue during delivery.

10. The system of claim 1, wherein the anchor comprises a textured portion of the delivery catheter and the anchoring system further comprises a retractable sheath configured to be translated relative to the delivery catheter to either (i) uncover the anchor for frictional engagement with the patient anatomy or (ii) cover the anchor to prevent frictional engagement with the patient anatomy.

11. The system of claim 2, wherein the anchoring system includes a plurality of anchors, each of the plurality of anchors including a base section and a gripping section, the gripping section including a plurality of frictional elements, wherein the plurality of anchors are shape-set to the compressed or stowed configuration such that, upon application of tension from the at least one control wire, the anchor is expanded from the compressed or stowed condition to the expanded or deployed configuration.

12. The system of claim 11, wherein the frictional elements, in the expanded or deployed configuration, are sized and shaped to frictionally engage with the patient anatomy to anchor the delivery system relative to the patient anatomy.

13. The system of claim 12, wherein the frictional elements are angled in an antegrade direction relative to the gripping section so as to prevent the frictional elements from inadvertently snaring tissue during delivery.

14. The system of claim 2, wherein the anchor comprises a single helix formed of a single wire or a multi-filament wire.

15. The system of claim 14, wherein the anchor comprising the single helix formed of the single wire or the multi-filament wire defines a first end, a second end, and a coiled section between the first end and the second end, wherein the anchor is shape-set to the expanded or deployed configuration such that, upon application of tension from the at least one control wire, the anchor transitions from the expanded or deployed condition to the compressed or stowed configuration.

16. A method of implanting a collapsible and expandable prosthetic heart valve into a patient, the method comprising:
advancing a delivery catheter, while the prosthetic heart valve is in a collapsed condition and mounted on a balloon of the delivery catheter, through vasculature of the patient until the collapsed prosthetic heart valve is positioned adjacent to or within a native valve annulus of the patient;
while the collapsed prosthetic heart valve is positioned adjacent to or within the native valve annulus, anchoring the delivery catheter to the vasculature to restrict axial movement of the delivery catheter relative to patient anatomy; and
inflating the balloon, while the delivery catheter is anchored to the vasculature, to expand the collapsible and expandable prosthetic heart valve for deployment into the native valve annulus.

17. The method of claim 16, wherein anchoring the delivery catheter includes expanding or deploying an anchoring system to engage with the vasculature.

18. The method of claim 17, further comprising applying or releasing tension on or more control wires that are coupled to an anchor of the anchoring system to expand or deploy the anchor of the anchoring system.

19. The method of claim 17, wherein the anchoring system is in a compressed or stowed configuration during advancing of the delivery catheter.

20. The method of claim 19, further comprising:
releasing the anchoring system from the patient anatomy after the prosthetic heart valve is deployed in the native valve annulus.
